(19) United States
(12) Patent Application Publication
(10) Pub. No.: US 2003/0152621 A1
(43) Pub. Date: Aug. 14, 2003

(54) Pharmaceutical formulation of gepirone for oral administration

(76) Inventors: J. G. J. Egberink, Oss (NL); J. F. Engelhart, West Orange, NJ (US)

Correspondence Address: INTERVET INC, 405 STATE STREET, PO BOX 318, MILLSBORO, DE, 19966, US

(21) Appl. No.: 10/054,458

(22) Filed: Nov. 21, 2001

(51) Int. Cl. A61K 9/22; A61K 9/14

(52) U.S. Cl. 424/468

(57) ABSTRACT

This invention makes a pharmaceutical formulation for oral administration with extended release properties available comprising an amount of gepirone hydrochloride, an amount of a cellulosic polymer matrix and an amount of microcrystalline cellulose characterised in that the amount the pharmaceutically acceptable cellulosic polymer matrix is from 70 to 85 wt %, the amount of carbohydrate binder is from 7 to 10 wt % and the amount of gepirone hydrochloride is from 13 to 21 wt %. The formulation is useful for a treatment for depression or a related central nervous system disorder with gepirone administered in a once-a-day oral formulation for extended release of gepirone.

The invention relates to a pharmaceutical formulation for oral administration with extended release properties comprising an amount of gepirone hydrochloride, an amount of a cellulosic polymer matrix and an amount of microcrystalline cellulose.

Such pharmaceutical formulations are described in EP 700 680 for use in the treatment of CNS disorders such as anxiety, depression and panic disorders. Gepirone is a drug which induces undesirable effects when high peak levels are reached. An even distribution of drug levels in blood is desirable to have a suitably tolerated therapeutic dose level. It was reported that optimal treatment of major depressive disorder was obtained with daily doses of up to 100 mg gepirone HCl (Wilcox et al; Psychopharmacology Bulletin). The administration of the total daily amount of gepirone was spread over the day by providing 20 mg gepirone HCl extended release tablets at intervals during the day. This was done not only because it was indicated in the prior art (EP 700 680) that extended release formulations can reliably be produced and have slow release properties when the content is at most 12% gepirone HCl of the total content, with a preferred maximum of 11 wt %, and the content of microcrystalline cellulose at least 10% with a preferred minimum of 11 wt %, but also because it could be expected that once-a-day high doses will be badly tolerated even when given as an extended release formulation.

Contrary to what is to be expected it was found that suitable higher strengths extended release tablets can be prepared and are well tolerated in a once a day dosage form in those patients already habituated to lower dosages.

Clearly, it is more accommodating for such patients to have a once-a-day formulation available.

This invention makes a pharmaceutical formulation according to the opening paragraph available in which the amount of the pharmaceutically acceptable cellulosic polymer matrix is from 70 to 85 wt %, the amount of carbohydrate binder is from 7 to 10 wt % and the amount of gepirone hydrochloride is from 13 to 21 wt %. The formulation may optionally contain further pharmaceutically acceptable additives, such as glidants, lubricants and colorants.

The invention makes a once per day medical treatment available with gepirone HCl in a pharmaceutical formulation for oral administration having the above-defined composition. This treatment is useful and well-tolerated by those patients treated for depression or a related central nervous system disorder, who are started on a treatment regime beginning with doses of about 20 mg gepirone HCl per day, and which is gradually built up to 60-100 mg gepirone HCl per day.

A pharmaceutical formulation for oral administration is usually a tablet or a capsule. Contrary to what would have been the weight of an 80 mg tablet according to EP 700 680, tablets according to the present invention can have a total weight of at most 450 mg. Despite the high relative amount of gepirone HCl over the cellulosic polymer matrix material and also over the carbohydrate binder, oral formulations, in particular tablets, could still be made with acceptable dissolution properties of gepirone and sufficient stability during production and handling.

A pharmaceutically acceptable cellulosic polymer matrix has the function of retaining gepirone HCl so that an extended release effect is obtained. Suitable matrixes include hydroxyalkylsubstituted alkylcelluloses having a viscosity of 15,000 cps to 100,000 cps. Hydroxymethyl propylcellulose (HPMC) of grades K15M and K100M is preferred and grade K100M, Premium (Methocel) is in particular preferred. The amounts of components in the pharmaceutical formulation of the invention are expressed as weight percentage (wt %) of the total weight of the formulation, which is usually a tablet. The term alkyl as used here means a branched or unbranched saturated unsubstituted carbon chain. In view of the required viscosities, the alkyl groups referred to in this paragraph do not comprise more than 6 carbon atoms.

The term pharmaceutically acceptable for suitable additives for use in carrying out the invention refers to requirements set for pharmaceutical auxiliaries in general. These requirements with regard to safety and non-interference with the active principle in pharmaceutical formulations are generally known to the skilled person. A standard compilation of pharmaceutically acceptable carriers and excipients can be found in the Handbook of Pharmaceutical excipients (2^{nd} edition edited by A. Wade and P. J. Weller; Published by the American Pharmaceutical Association, Washington and The Pharmaceutical Press, London in 1994). Additives to a pharmaceutical formulation, such as carriers, binders, glidants, lubricants and colorants are used for example in order to obtain certain cohesiveness, coloration and flowability of the tablets. In a preferred embodiment of this invention magnesium stearate, colloidal silicon dioxide and iron oxide pigments are used.

Glidants and lubricants are agents reducing the adhesiveness of the powder mixture or tablets during production. Methods of use of such additives are known in the art of making pharmaceutical compositions as for example described in chapter 19 of Remington's Pharmaceutical Sciences (18th edition Editor A. R. Gennaro; Mack Publishing Comp; Easton, Pa.).

Binders are agents used to impart cohesive properties to a pharmaceutical composition resulting in minimal loss from the pharmaceutical composition during production and handling. Carbohydrate binders are for example cellulose, methylcellulose, ethylcellulose, hydroxypropylcellulose, sugars, starches, amylopectin, dextrin, maltodextrin, gums and alginates. Microcrystalline cellulose, and in particular Avicel pH 101, is a preferred binder for use in this invention.

The term pharmaceutical formulation for oral administration with extended release properties is used in this description to refer to the characteristics of extended release of gepirone according to the disclosure in EP 700 680. Specifically, a formulation according to the invention has a release rate of gepirone from the formulation such that about 18 to 24 hours are required to attain from about 90 to about 95% absorption of gepirone.

Oral pharmaceutical formulations according to the present invention can be prepared by methods known in the art, e.g. as described in the standard reference, Gennaro et al., Remington's Pharmaceutical Sciences, (18th ed., Mack Publishing Company, 1990, see especially Part 8: Pharmaceutical Preparations and Their Manufacture). Some caution in handling the compressed high strength formulations of this invention is advisable in order to avoid breaking and cracks in tablets.

Gepirone may be prepared by any method known in the art. Typically the compound is prepared by the methods described in U.S. Pat. No. 4,423,049. Pharmaceutical extended release compositions containing gepirone are disclosed in EP 700 680. The contents of these documents are incorporated herein by reference.

The following examples will serve to illustrate how to perform the invention.

### EXAMPLE 1

Manufacturing Procedure of Batches of 160,000 Tablets

Composition of tablets (Numbers in the table are amounts in mg)

| Ingredients | 40 mg | 60 mg | 80 mg | Function |
|---|---|---|---|---|
| Gepirone HCl | 40.0 | 60.0 | 80.0 | Active |
| | | | | principle |
| Hydroxypropyl | 290.0 | 290.0 | 290.0 | Drug release |
| Methylcellulose (Methocel | | | | controlling |
| K100M, Premium) | | | | polymer |
| Microcrystalline | 52.12 | 31.0 | 33.7 | Diluent |
| Cellulose (Avicel pH 101) | | | | |
| Euroxide yellow or red | 0.08 | 1.2 | 3.5 | Colorant |
| (E7055, E7056 or E7016 | | | | |
| Colloidal silicon dioxide | 1.6 | 1.6 | 1.6 | Glidant |
| (cab-o-sil M5) | | | | |
| Magnesium stearate NF | 1.2 | 1.2 | 1.2 | Lubricant |

Active Pre-Mixture:

Transfer the colloidal silicon dioxide, NF, colorant (40 mg: Euroxide Yellow E 7056; 60 mg: Euroxide Yellow E 7055; 80 mg: Euroxide yellow E 7055 and Euroxide Red E 7016), gepirone HCl powder and 20% of hydroxypropyl methylcellulose USP in 2 cu. Ft. planetary mixer (Hobart mixer). Mix ingredients for 15 minutes in a planetary mixer (Hobart Mixer). Label as ‘Active Pre-Mix’.

Blend for Slugging

Mill the Active Pre-Mix in a Fitzmill using a perforated plate No. 0020 at high speed, impact forward to deagglomerate lumps, if any.

Transfer the Active Pre-Mix in a 10 cu. Ft. “V-blender without an I-bar, while passing through # 12 mesh screen and transfer the balance of 80% HPMC, microcrystalline cellulose, NF and 50% of magnesium stearate, NF in the V-blender without an I-bar. Blend ingredients in the V-blender without an I bar for 24 minutes and label as “Blend for Slugging”.

Slugging

Compress the blend into slugs using ⅞″ round flat face bevelled edge (40) bevelled plain (60, 80) tooling using a rotary Kikusui-Hercules compression machine.

In Process Controls:

| | | |
|---|---|---|
| Weight: | 2250 | mg |
| Hardness: | 7 | kp |

| | |
|---|---|
| Targeted thickness: | 0.255″ |

Final Blend

Mill the slugs in an S. S. Fitzmill with screw feeder using a perforated plate No. 0093 at medium speed, knives forward and screw feeder setting of 3.5±0.5. Transfer the milled mass into a 10 cu. Ft. S.S. V-blender without I-bar. Screen the balance of 50% magnesium stearate, NF through # 18 mesh and transfer also into the V-blender. Blend for 6 minutes.

Compress tablets with a rotary Kikusui-Libra compression machine using 0.338″×0.405″ Ovoid rectangular dies.

In Process Controls:

| Strength, mg | 40 & 60 | 80 |
|---|---|---|
| Run Weight, mg | 385 ± 27 | 410 ± 29 |
| Hardness, kp | 18 ± 4 | 20 ± 8 |
| Thickness, inches | 0.230 to 0.260 | 0.235 to 0.265 |

Store in tight containers untill further use or testing.

Drug release profile of tablets prepared as described in this example. Additional 20 mg tablets were made according to the procedure described in EP 700 680 for comparison of the drug release profile of the 60 and 80 tablets according to this invention:

Gepirone release pattern of tablets (Numbers in the table represent the
percentage dissolution of the theoretical content of the tablets

| Released after: → | 1 hr | 5 hr | 12 hr | 20 hr | recovery |
|---|---|---|---|---|---|
| 20 mg tablets | 19 | 49 | 77 | 93 | 102 |
| 40 mg tablets | 21 | 50 | 77 | 93 | 100 |
| 60 mg tablets | 21 | 51 | 81 | 97 | 102 |
| 80 mg tablets | 22 | 52 | 80 | 96 | 101 |

### EXAMPLE 2

For upscaling the manufacturing for batches of 800,000 tablets certain adaptations were applied:

Thus, procedures are according to the methods described in example 1, but the active pre-mix is blended in a 340 qt. AZMF Glen mixer for 28 minutes, the size of the V-blender used is 30 cu. Ft. and final blending is done for 7 minutes. Tablets had similar properties as measured in example 1. Special attention is given to the problem of friability of the 80 mg strength tablets. It is preferred to compress 80 mg tablets at a speed of the compression machine of 20 rotations per minute, i.e. in general at a speed of less than 30 rpm, set at the tablet compression machine in order to reduce the number of tablets with cracks.

1. An extended release gepirone hydrochloride pharmaceutical formulation for oral administration comprising cellulosic polymer matrix as 70 to 85 wt%, carbohydrate binder as 7 to 10 wt % and gepirone hydrochloride as 13 to 21 wt %.

2. The pharmaceutical formulation according to claim 1, wherein the cellulosic polymer matrix is a hydroxymethyl propylcellulose having a viscosity of 15,000 cps to 100,000 cps.

3. The pharmaceutical formulation according to claim 1, comprising Avicel pH 101.

4. A method for treating a central nervous system disorder comprising administering an effective amount of a once-a-day extended release oral formulation of gepirone hydrochloride according to claim 1.

5. The method of claim 4, wherein the central nervous system disorder is depression.
